(19) United States
(12) Patent Application Publication
(10) Pub. No.: US 2008/0260785 A1
(43) Pub. Date: Oct. 23, 2008

(54) PAROXETINE COMPOSITIONS

(75) Inventors: Raghupathi Kandarapu, Hyderabad (IN); Vijay Dinanathji Nasare, Nagpur (IN); Indu Bhushan, Hyderabad (IN); Mailatur Sivaraman Mohan, Hyderabad (IN); Nagaraju Nagesh, Hyderabad (IN)

Correspondence Address: DR. REDDY'S LABORATORIES, INC., 200 SOMERSET CORPORATE BLVD, SEVENTH FLOOR,, BRIDGEWATER, NJ, 08807-2862, US

(73) Assignees: DR. REDDY'S LABORATORIES LIMITED, Hyderabad 500 016, Andhra Pradesh (IN); DR. REDDY'S LABORATORIES, INC., Bridgewater, NJ (US)

(21) Appl. No.: 12/051,589

(22) Filed: Mar. 19, 2008

Related U.S. Application Data
(63) Continuation-in-part of application No. PCT/US2006/036654, filed on Sep. 20, 2006.
(60) Provisional application No. 60/718,788, filed on Sep. 20, 2005.

(51) Int. Cl. A61K 9/00; A61K 31/4525

(52) U.S. Cl. 424/400; 514/321

(57) ABSTRACT

The invention relates to pharmaceutical compositions having polymers that release paroxetine in a controlled manner for a prolonged or sustained period of time. An embodiment of the invention provides enhanced bioavailability controlled release pharmaceutical compositions comprising paroxetine or its pharmaceutically acceptable salts, which enables a reduction in its orally administered dose.

The present invention relates to controlled release pharmaceutical compositions comprising paroxetine or pharmaceutically acceptable salts, solvates, polymorphs, enantiomers or mixtures thereof. More particularly this invention relates to pharmaceutical compositions having one or more polymers that release paroxetine in a controlled manner for a prolonged or sustained period of time.

Further this invention also relates to controlled release pharmaceutical compositions comprising paroxetine and one or more polymers, which result in improved bioavailability of paroxetine in human subjects, thus enabling a reduction in its orally administrable dose.

Paroxetine has a chemical name (−)-trans-4R-(4′-fluorophenyl)-3S-[(3′,4′-methylenedioxyphenoxy)methyl]piperidine, and in the form of its hydrochloride hemihydrate is an odorless, off-white powder, having a melting point range of 120° C. to 138° C. and a solubility of 5.4 mg/ml in water. The structural formula for paroxetine hydrochloride is Formula I.

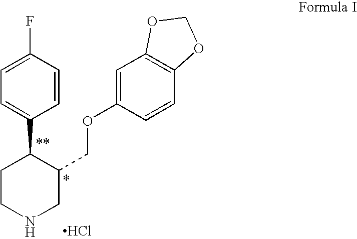

Paroxetine is useful in the treatment of major depressive disorder and is commercially available in enteric-coated controlled release tablets (PAXIL® CR) manufactured by GlaxoSmithKline, containing 12.5 mg, 25 mg or 37.5 mg of paroxetine equivalent, the drug being present in the form of paroxetine hydrochloride hemihydrate.

Controlled release (“CR”) drug delivery systems are useful in delivering active pharmaceutical ingredients that have a narrow therapeutic range, short biological half-life and/or high toxicities. These systems allow the dosage delivery by reducing the number of administrations and provide the desired therapeutic effect throughout the day.

U.S. Pat. Nos. 4,839,177, 5,422,123 and 6,548,084 describe controlled release formulations of paroxetine.

U.S. Pat. No. 6,350,471 discloses a delayed release tablet comprising a core containing paroxetine.

U.S. Patent Application Publication Nos. 2006/0039975, 2005/0059701 and 2005/0266082, and International Application Publication Nos. WO 2005/107716 and WO 2005/034954, disclose controlled release compositions comprising paroxetine.

U.S. Patent Application Publication No. 2004/0224960 describes a method of enhancing bioavailability of paroxetine (a substrate for cytochrome P450 enzyme) using a cytochrome P450 enzyme-inhibiting amount of a compound that decreases paroxetine metabolism in mammals.

Major commonly observed adverse effects associated with PAXIL® CR tablets include gastrointestinal bleeding, nausea, dizziness, headache, infection, dry mouth, vomiting, abnormal vision, abnormal ejaculation, diarrhea, constipation, sweating, trauma, tremor and yawning. A risk of suicidal behavior has also been reported in adolescents taking paroxetine. Most of these adverse effects are related to high exposure of drug and variations in paroxetine plasma concentrations.

A controlled release pharmaceutical composition of paroxetine with one or more polymers resulting in enhanced bioavailability, thus enabling reduction in dose, will be a significant improvement in the field of solid oral therapeutic compositions.

### SUMMARY OF THE INVENTION

An aspect of the invention provides controlled release pharmaceutical compositions comprising:

a core comprising paroxetine or a pharmaceutically acceptable salt and a polymer that releases paroxetine in a controlled manner; and

a coating over the core comprising an acid-resistant polymer.

An embodiment of a pharmaceutical composition releases at least about 60 percent of a total contained paroxetine within about 4 hours, and at least about 90 percent of a total contained paroxetine within about 8 hours, during immersion in an aqueous fluid having a pH about 7.5, at body temperature, using a USP type 2 dissolution test apparatus.

In an embodiment, a pharmaceutical composition produces, upon administration of a single dose containing 37.5 mg paroxetine equivalent to a human, values of paroxetine Cₘₐₓ and AUC in plasma at least about twice the values obtained after administration of the commercial product PAXIL® 37.5 mg paroxetine CR tablets.

A further aspect of the invention provides controlled release pharmaceutical compositions comprising reduced doses of paroxetine or its pharmaceutically acceptable salts; wherein enhanced bioavailability of said pharmaceutical compositions enables a significant reduction in the orally administered dose without compromising its therapeutic benefit.

In an embodiment of the present invention, one or more polymers modulates the release of paroxetine in a controlled manner for a prolonged or sustained period of time.

In another embodiment, e pharmaceutical compositions of present invention release at least about 80 percent of a total contained paroxetine within about 6 hours during immersion in an aqueous fluid having a pH about 7.5, at body temperature, using a USP type 2 dissolution test apparatus.

In an embodiment, the invention provides, upon administration of a single dose containing 37.5 mg of paroxetine to human subjects, a mean paroxetine Cₘₐₓ at least about 15 ng/ml and a mean AUC at least about 400 ng-hour/ml.

An embodiment of the invention provides controlled release paroxetine compositions wherein enhanced bioavailability enables significant reduction in the orally administrable dose without compromising its therapeutic benefit.

In an embodiment, the invention provides pharmaceutical compositions comprising:

a) a compressed core containing a mixture comprising:
paroxetine or a salt thereof, ethylcellulose, and a hydroxypropyl methylcellulose polymer having a nominal viscosity about 5 to about 100 cP, or
paroxetine or a salt thereof and a combination of a hydroxypropyl methylcellulose polymer having a nominal viscosity about 25,000 to about 100,000 cP and a hydroxypropyl methylcellulose polymer having a nominal viscosity about 5 to about 100 cP; and

b) a coating over the core comprising an acid-resistant polymer.

In another embodiment, the invention provides pharmaceutical compositions comprising:

a compressed core containing paroxetine or a salt thereof, a hydroxypropyl methylcellulose polymer, and an ethylcellulose polymer; and

a coating over the core comprising an acid-resistant polymer.

In a further embodiment, the invention provides pharmaceutical compositions comprising:

a compressed core containing paroxetine or a salt thereof and a combination of a hydroxypropyl methylcellulose polymer having a nominal viscosity about 100,000 cP and a hydroxypropyl methylcellulose polymer having a nominal viscosity about 15 cP; and

a coating over the core comprising an acid-resistant polymer.

In an embodiment, the invention provides pharmaceutical compositions comprising:

a compressed core containing a mixture comprising paroxetine or a salt thereof and a combination of a hydroxypropyl methylcellulose polymer having a nominal viscosity about 4,000 to about 15,000 cP and a hydroxypropyl methylcellulose polymer having a nominal viscosity about 5 to about 100 cP; and

a coating over the core comprising an acid-resistant polymer.

In another embodiment, the invention provides pharmaceutical compositions comprising:

a compressed core containing paroxetine or a salt thereof and a combination of a hydroxypropyl methylcellulose polymer having a nominal viscosity about 4,000 cP and a hydroxypropyl methylcellulose polymer having a nominal viscosity about 100 cP; and

a coating over the core comprising an acid-resistant polymer.

In a further embodiment, the invention provides pharmaceutical compositions comprising:

a compressed core containing paroxetine or a salt thereof and a combination of two hydroxypropyl methylcellulose polymers having nominal viscosity about 4,000 to about 15,000 cP; and

a coating over the core comprising an acid-resistant polymer.

In a still further embodiment, the invention provides pharmaceutical compositions comprising:

a compressed core containing paroxetine or a salt thereof and a combination of a hydroxypropyl methylcellulose polymer having a nominal viscosity about 4,000 to about 15,000 cP and a hydroxypropyl methylcellulose polymer having a nominal viscosity about 25,000 to about 100,000 cP; and

a coating over the core comprising an acid-resistant polymer.

In a yet further embodiment, the invention provides pharmaceutical compositions comprising:

a compressed core containing paroxetine or a salt thereof and a combination of hydroxypropyl methylcellulose polymers comprising about 5 to about 10 weight percent of a hydroxypropyl methylcellulose polymer having a nominal viscosity about 4,000 to about 15,000 cP and about 4 to about 15 weight percent of a hydroxypropyl methylcellulose polymer having a nominal viscosity about 5 to about 100 cP; and

a coating over the core comprising an acid-resistant polymer.

### DETAILED DESCRIPTION OF THE INVENTION

The present invention relates to controlled release pharmaceutical compositions comprising paroxetine or pharmaceutically acceptable salts, solvates, polymorphs, enantiomers or mixtures thereof. More particularly, this invention relates to pharmaceutical compositions having one or more polymers that release paroxetine in a controlled manner for a prolonged or sustained period of time.

Further this invention also relates to controlled release pharmaceutical compositions comprising paroxetine and one or more polymers, which result in improved bioavailability of paroxetine in human subjects, thus enabling significant (for example, about 50 percent) reduction in its orally administrable dose.

The present invention utilizes one or more polymers, including a mixture of different polymers, to modulate the release of the paroxetine in a controlled manner for a prolonged or sustained period of time.

A majority of adverse effects of paroxetine are related to its high exposure to the gastrointestinal tract and fluctuations in peak and trough plasma concentrations, and this has remained the case after introduction of the controlled release formulation (PAXIL® CR tablets) to the market. Hence, improving the bioavailability of paroxetine and thus reducing the dose to get same therapeutic benefit will be one of the ideal approaches to minimize the adverse effects of paroxetine.

Surprisingly, it has been observed that a controlled release pharmaceutical composition in accordance with an embodiment of the invention comprising paroxetine, at least one release retarding polymer and an acid resistant coating showed a comparable in vitro dissolution profile against the commercial product (PAXIL® 37.5 mg CR tablets), but resulted in more than two-fold higher values of Cₘₐₓ and AUC when administered to human subjects. Variability in in vitro and in vivo data (intra- and inter-subject variability) has also been reduced significantly. This significant improvement in bioavailability can lead to marked reduction in the dose of paroxetine and thus minimize related adverse effects.

A controlled release pharmaceutical composition of the present invention results in a surprising and significant improvement of oral bioavailability of paroxetine, enabling significant reduction in its orally administrable dose without compromising its pharmacokinetic parameters (Cₘₐₓ and AUC), and thus, the therapeutic benefit. The term “Cₘₐₓ” is commonly used to identify the maximum concentration of drug in plasma that is achieved, following administration of a drug dose. “AUC” is the commonly used term representing the area under a plot of drug concentrations in plasma versus the elapsed time after administration of a drug dose.

In one embodiment, this invention provides for a pharmaceutical composition having one or more polymers that release paroxetine in a controlled manner for a prolonged or sustained period of time.

In another embodiment, the present invention provides for reduction in the administered dose of paroxetine because of significant improvement in bioavailability, when it is formulated in a controlled release composition that is coated with an acid-resistant (enteric) coating material of a defined coating build-up to prevent the release of paroxetine in acidic environments. The system comprises one or more hydrophilic polymers, or one or more hydrophobic polymers, or a mixture of hydrophilic and hydrophobic polymers, wherein the rate of release of paroxetine is controlled via a monolithic matrix composition, or a reservoir composition, or combinations thereof.

An aspect of the present invention provides paroxetine compositions with reduced dose that provide comparable pharmacokinetic parameters (Cₘₐₓ and AUC), vis-à-vis marketed PAXIL® CR tablets when tested in humans under identical conditions. Thus paroxetine compositions of the present invention, with a significantly reduced dose, can show a similar therapeutic benefit to that of PAXIL® CR tablets.

The “reduced dose compositions” of the present invention refer to pharmaceutical compositions comprising a lesser amount of paroxetine or its pharmaceutically acceptable salts per unit dose, as compared to the marketed PAXIL® CR tablets, for achieving a similar therapeutic effect.

An embodiment of the invention provides, upon administration of a single dose containing 37.5 mg of paroxetine to human subjects, a mean paroxetine Cₘₐₓ at least about 15 ng/ml and a mean AUC at least about 400 ng-hour/ml.

An embodiment of the present invention provides monolithic matrix compositions comprising various hydrophilic polymers having a high degree of swelling in aqueous fluids, or hydrophobic polymers, either alone or in mixtures thereof, wherein the rate of drug release is primarily controlled by diffusion and erosion. Whereas, in the case of a reservoir composition, the rate of drug release is primarily controlled by diffusion of drug through a release retarding membrane barrier comprising a hydrophilic or hydrophobic polymer, either alone or in mixtures thereof.

In one aspect of the present invention, the pharmaceutical compositions of paroxetine comprise a core and a coating on it. The core further comprises active ingredient and one or more polymers, whereas the coating comprises a pH sensitive polymer.

Useful hydrophilic polymers of various grades include, but are not limited to: cellulose derivatives such as methylcellulose, carboxymethyl cellulose, hydroxypropyl methylcellulose, cross-linked sodium carboxymethyl cellulose, and cross-linked hydroxypropyl cellulose; carboxymethylamide; potassium methacrylate/divinylbenzene copolymers; polymethylmethacrylate; polyhydroxyalkyl methacrylate; cross-linked polyvinylpyrrolidone; high-molecular weight polyvinylalcohols; gums such as natural gum, agar, agrose, sodium alginate, carrageenan, fucoidan, furcellaran, laminaran, hypnea, eucheums, gum arabic, gum ghatti, gum karaya, gum tragacanth and locust bean gum; hydrophilic colloids such as alginates, carbopol and polyacrylamides; other substances such as arbinoglactan, pectin, amylopectin, gelatin, N-vinyl lactams, polysaccharides; and the like. Combinations of any two or more of these polymers, and other polymers having the required properties are within the scope of the invention.

Hydroxypropyl methylcellulose polymers (also called “hypromellose”) can be defined chemically as partially O-methylated and partially O-(2-hydroxypropylated) cellulose. Among the products used in pharmaceutical products are those described in the “USP” monograph from United States Pharmacopeia 24, United States Pharmacopeial Convention, Inc., Rockville, Md. (1999) at pages 843-844:

| | | |
|---|---|---|
| Chemical | % Methoxy | % Propoxy |

| Type | Minimum | Maximum | Minimum | Maximum |
|---|---|---|---|---|
| 1828 | 16.5 | 20.0 | 23.0 | 32.0 |
| 2208 | 19.0 | 24.0 | 4.0 | 12.0 |
| 2906 | 27.0 | 30.0 | 4.0 | 7.5 |
| 2910 | 28.0 | 30.0 | 7.0 | 12.0 |

Commercial products are available in various grades, characterized by their viscosities at 20° C. in 2 percent (w/v) aqueous solutions. Some of the METHOCEL™ products that are available from Dow Chemical Company, Midland, Mich. U.S.A. are listed in the following table:

| Product | Chemical Type | Nominal Viscosity (cP*) |
|---|---|---|
| E6 | 2910 | 6 |
| E15 | 2910 | 15 |
| E4M | 2910 | 4,000 |
| K100 | 2208 | 100 |
| K4M | 2208 | 4,000 |
| K15M | 2208 | 15,000 |
| K100M | 2208 | 100,000 |

*cP is centipoise, as determined using the USP Test 911 procedure mentioned in the USP monograph.

The available nominal viscosities from this supplier for the different chemical types range from about 2.4 to 100,000 cP. Other suppliers of hypromellose products with various viscosities include Hercules, Inc. of Wilmington, Del. U.S.A., and these products are sold using the BENECEL trademark. All hydroxypropyl methylcellulose viscosities specified herein are for 2 percent (w/v) aqueous solutions, and are determined using the USP Test 911 procedure at 20° C. Viscosity results obtained using other techniques, such as the Brookfield™ viscometer or the European Pharmacopeia method, typically differ from the USP method results.

Useful hydrophobic polymers or combinations thereof used in various ratios include, but are not limited to, celluloses such as methyl cellulose, ethyl cellulose, low-substituted hydroxypropylcellulose (L-HPC), cellulose acetates and their derivatives, cellulose acetate phthalate, hydroxypropyl methylcellulose phthalate, cellulose acylate, cellulose diacylate, cellulose triacylate, cellulose acetate, cellulose diacetate, cellulose triacetate, mono-, di- and tri-cellulose alkanylates, mono-, di-, and tri-cellulose arylates, and mono-, di- and tri-cellulose alkenylates, crosslinked vinylpyrrolidone polymers (also called “crospovidone”), glyceryl behenate, polymethacrylic acid based polymers and copolymers sold under the trade name of EUDRAGIT™ (including Eudragit RL and RS, NE-30D), zein, and aliphatic polyesters. Other classes of polymers, copolymers of these polymers or their mixtures in various ratios and proportions as required are within the scope of this invention without limitation.

Of course, any other polymers, which demonstrate similar hydrophobic characteristics, are also acceptable in the working of this invention.

In one of the embodiments, polymers simultaneously possessing swelling and gelling properties, such as hydroxypropyl methylcellulose, have been found particularly useful in either alone or in combination with a hydrophobic polymer such as ethylcellulose, to modulate the release of the drug paroxetine in a predictable controlled manner for a prolonged or sustained period of time.

According to the present invention, the concentration of hydrophilic polymers and/or hydrophobic polymer ranges from about 5% to 90% of the total weight of the paroxetine-containing core, an individual hydrophilic polymer typically being present at about 5 to about 45 percent by weight.

In certain embodiments when mixtures of hydrophilic polymers are used, one frequently have a high viscosity, such as about 25,000 to about 100,000 cP, or higher, and the other will have a lower viscosity, such as about 5 to about 100 cP. In a specific embodiment, the high viscosity hypromellose can have a viscosity about 100,000 cP and the low viscosity hypromellose can have a viscosity about 15 cP.

In further embodiments, when mixtures of hydrophilic polymers are used, one will have a medium viscosity, such as about 4,000 to about 15,000 cP, or higher, and the other will have a low viscosity, such as about 5 to about 100 cP. In a specific embodiment, the medium viscosity hypromellose can have a viscosity about 4,000 cP and the low viscosity hypromellose can have a viscosity about 100 cP. Specific embodiments of pharmaceutical formulations comprise a compressed core containing paroxetine or a salt thereof and a combination of hydroxypropyl methylcellulose polymers comprising about 5 to about 10 weight percent of a hydroxypropyl methylcellulose polymer having a nominal viscosity about 4,000 to about 15,000 cP and about 4 to about 15 weight percent of a hydroxypropyl methylcellulose polymer having a nominal viscosity about 5 to about 100 cP. In some instances, hydroxypropyl methylcellulose polymers having nominal viscosities about 4,000 cP and about 100 cP will be used for these specific embodiments.

In further embodiments, when a mixture of two hydrophilic polymers is used, both will have a medium viscosity, such as about 4,000 to about 15,000 cP, or higher. In specific embodiments, the first medium viscosity hypromellose can have a viscosity about 4,000 cP and the second medium viscosity hypromellose can have a viscosity about 10,000 cP.

In further embodiments, when mixtures of hydrophilic polymers are used, one will have a medium viscosity, such as about 4,000 to about 15,000 cP, or higher, and the other will have a high viscosity, such as about 25,000 to about 100,000 cP, or higher.

In an embodiment, the paroxetine-containing core comprises a combination of a hydrophobic polymer and a hydrophilic polymer. Frequently, the core will comprise about 10 to about 45 weight percent of the hydrophilic polymer and about 10 to about 45 weight percent of the hydrophobic polymer. The hydrophilic polymers frequently will be in the low viscosity range, such as those hypromellose polymers having nominal viscosities by the USP Test 911 procedure between about 5 and about 100 cP. Useful hydrophobic polymers include ethylcellulose, ethyl ethers of cellulose, for which various viscosity grades are available under the trademark ETHOCEL from Dow Chemical Company, Midland, Mich. U.S.A. In many instances, the higher viscosity products, such as those having nominal viscosities of about 45 to about 100 cP, will be used, this viscosity being determined using the USP Test 911 procedure at 25° C., in accordance with the ethylcellulose monograph in The National Formulary, 19^{th} Ed., United States Pharmacopeial Convention, Inc., Rockville, Md. U.S.A. (1999) at page 2451.

When used in combination, the weight ratio of the hydrophilic to hydrophobic polymer materials ranges from about 1:9 to 9:1, respectively.

An embodiment of the invention includes a core comprising paroxetine, ethylcellulose, and hypromellose. The ethylcellulose can have a viscosity about 45 to about 100 cP and the hypromellose can have a viscosity about 5 to about 100 cP. A specific embodiment utilizes ethylcellulose having a 100 cP viscosity and hypromellose having a 15 cP viscosity.

The pharmaceutical compositions of the present invention may further contain one or more diluents to makeup the tablet mass so that it becomes easier for the patient and the caregiver to handle. Common suitable diluents are microcrystalline cellulose, micro fine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, potassium chloride, powdered cellulose, sodium chloride, sorbitol, talc and the like.

The pharmaceutical compositions to be made into tablets may further include a disintegrant to accelerate disintegration of the tablet in the patient's stomach. Useful disintegrants include but are not limited to alginic acid, carboxymethyl cellulose calcium, carboxymethylcellulose sodium (e.g. Ac-Di-Sol®, Primellose®), colloidal silicon dioxide, croscarmellose sodium, crospovidone (e.g. Kollidon®, Polyplasdone®), guar gum, magnesium aluminum silicate, methyl cellulose, microcrystalline cellulose, polacrilin potassium, powdered cellulose, pregelatinized starch, sodium alginate, sodium starch glycolate (e.g. Explotab®), and starch.

Various materials that may be used as acid-resistant polymers include, but are not limited to, cellulose acetate butyrate, cellulose acetate phthalate; hydroxypropyl methylcellulose phthalate, hydroxypropyl methylcellulose acetate phthalate, copolymers of methacrylic acid and methacrylates (Eudragit®); polyalkyl acrylates; polyvinyl acetate phthalate; chitosan; crosslinked vinylpyrrolidone polymers; and the like. Other classes of acid-resistant coating or their mixtures in various ratios as required are also within the purview of this invention without limitation.

Optionally, pharmaceutical compositions of present invention may have an outermost non-functional film coating comprising materials such as carboxymethyl cellulose sodium, hydroxyethyl cellulose, hydroxypropyl methylcellulose (HPMC); and the like. Such coatings are commonly used to improve the aesthetics of a dosage form and can provide a suitable surface for imprinting.

Plasticizers that can be used in coatings include, without limitation, acetyltributyl citrate, phosphate esters, phthalate esters, amides, mineral oils, fatty acids and esters, glycerin, triacetin or sugars, fatty alcohols, polyethylene glycol, ethers of polyethylene glycol, fatty alcohols such as cetostearyl alcohol, cetyl alcohol, stearyl alcohol, oleyl alcohol, myristyl alcohol and the like.

Pharmaceutical compositions for tableting and film formation may further include additional components, such as, but not limited to, pharmaceutically acceptable glidants, lubricants, flavoring agents, opacifiers, colorants, and other commonly used excipients.

Solvents that can be used in processing include, but are not limited to: aqueous solvents such as water; organic volatile solvents such as acetaldehyde, acetone, benzene, carbon disulphide, carbon tetrachloride, 1,2 dichloroethane, dichloromethane, N,N-dimethylformamide, 1,4-dioxane, epichlorhydrin, ethyl acetate, ethanol, ethyl ether, ethylene glycol, 2-ethoxyethanol (acetate), formaldehyde, isopropanolol, methanol, methyl n-butyl ketone, methyl ethyl ketone, 2-methoxyethanol (acetate), perchloroethylene, toluene, 1,1,1-trichloroethane, trichloroethylene; and the like.

In one embodiment, a controlled release paroxetine composition of the present invention comprises a mixture of paroxetine, two or more hydroxypropyl methylcelluloses having different grades of viscosities, glyceryl behenate, one or more surfactants and other pharmaceutically acceptable additives, said composition being coated with a pH-dependent methacrylate copolymer that forms acid resistant films, and optionally an outermost non-functional film coating, such composition exhibiting marked (such as about two-fold) enhancement in oral bioavailability parameters.

The present invention provides for a unit dose of paroxetine of about 5 to about 50 milligrams, or about 6 to about 30 milligrams, per dosage form.

In one embodiment, the controlled release compositions are prepared by wet granulation without the use of a binder.

In another embodiment, a controlled release composition is additionally coated with an acid-resistant coating material to a defined coating build-up to prevent the release of paroxetine in acidic environments.

The hydrophilic-hydrophobic swellable monolithic compositions are stable during storage. They show low inter- and intra-individual variability. Also the compositions give a generally linear initial dissolution profile.

The hydrophilic-hydrophobic swellable monolithic composition along with other pharmaceutically acceptable excipients are formulated into a suitable solid oral dosage form such as tablets and the like, by procedures known to a person skilled in the art of preparation of pharmaceutical formulations. Such compositions can include other excipients as are required for the preparation of the compositions, including but not limited to diluents, granulating agents, solvents, lubricants, wetting agents, disintegrating agents and the like.

The following examples will further describe certain specific aspects and embodiments of the invention in greater detail, are provided only for the purpose of illustration, and are not intended to limit the scope of the invention.

### EXAMPLE 1

### Compositions for Paroxetine CR Tablets (12.5, 25, and 37.5 mg Paroxetine)

| | Quantity for 1000 Tablets (g) | | | | | |
|---|---|---|---|---|---|---|
| | 12.5 mg | | 25 mg | | 37.5 mg | |
| Ingredient | Strength | | Strength | | Strength | |
| Paroxetine hydrochloride | 14.6 | | 29.2 | | 43.8 | |
| hemihydrate | | | | | | |
| Ethylcellulose 100 cP | 90 | | 90 | | 90 | |
| Hydroxypropyl methylcellulose 15 cP | 90 | | 90 | | 90 | |
| (Methocel ™ E15) | | | | | | |
| Tricalcium phosphate | 37.9 | | 27.8 | | 23.2 | |
| Magnesium stearate | 2.5 | | 3 | | 3 | |
| Core weight | 235 | mg | 240 | mg | 250 | mg |
| Eudragit L100-55* | 16.8 | | 17.6 | | 18.4 | |
| Triethyl citrate | 1.7 | | 1.8 | | 1.8 | |
| Talc | 2.5 | | 2.6 | | 2.8 | |
| Isopropyl alcohol | 560 | | 600 | | 600 | |
| Finished tablet weight | 256 | mg | 262 | mg | 273 | mg |

*EUDRAGIT ™ L100-55 is a pH-dependent methacrylate copolymer that forms acid resistant films, which are not soluble below about pH 5.5, and is manufactured by Röhm & Co. GmbH of Darmstadt, Germany. The polymer is chemically described as poly(methacrylic acid), ethyl acrylate 1:1.

Manufacturing Process:

1. Paroxetine hydrochloride hemihydrate, ethylcellulose, hydroxypropyl methylcellulose 15 cP, tricalcium phosphate and magnesium stearate were sieved and mixed uniformly.

2. The dry blend was directly compressed into tablets using 8.5 mm round, biconcave punches to give a hardness of about 4-7 kP. (kP is “kilopond,” corresponding to kg force.)

3. Coating solution was prepared by dissolving Eudragit L 100-55 in isopropyl alcohol (8% w/w). Further, triethyl citrate and talc were added to the coating solution.

4. The core tablets were then coated with above coating solution until a weight buildup of 8 to 9% w/w was achieved.

### EXAMPLE 2

### Composition for Paroxetine 37.5 mg CR Tablets

| | Quantity | |
|---|---|---|
| | for 1000 | |
| Ingredient | Tablets (g) | |
| Paroxetine hydrochloride hemihydrate | 43.8 | |
| Ethylcellulose 100 cP | 34 | |
| Hydroxypropyl methylcellulose 15 cP | 64 | |
| (Methocel E15) | | |
| Glyceryl behenate | 40 | |
| Dicalcium phosphate | 19.5 | |
| Copovidone | 11 | |
| Colloidal silicon dioxide | 3.3 | |
| Sodium stearyl fumarate | 4.4 | |
| Core weight | 220 | mg |
| Eudragit L100-55 | 16.8 | |
| Triethyl citrate | 1.7 | |
| Talc | 2.5 | |
| Isopropyl alcohol | 600 | |
| Enteric coated tablet weight | 241 | mg |
| Opadry YS-1-106134* | 12.5 | |
| Finished tablet weight | 253.5 | mg |

*Opadry YS-1-106134 is ready mix film coating material from Colorcon, West Point, Pennsylvania U.S.A., containing hypromellose, titanium dioxide, macrogol and dark blue pigment.

Manufacturing Process:

1. Paroxetine hydrochloride hemihydrate and dicalcium phosphate were dry mixed and granulated with water, dried in fluid bed drier at a temperature 55-65° C. till the moisture content was 2% w/w when tested using an infrared moisture analyzer at a temperature of 105° C.

2. Dried granules were passed through a 60 mesh ASTM sieve.

3. Sifted granules were blended with ethylcellulose, hydroxypropyl methylcellulose 15 cP, glyceryl behenate, copovidone, colloidal silicon dioxide and sodium stearyl fumarate.

4. This blend was compressed into tablets (8.5 mm round, biconcave punches to have a hardness of about 4-7 kP).

5. Coating solution was prepared by dissolving Eudragit L 100-55 in isopropyl alcohol (8% w/w). Further, triethyl citrate and talc were added to the coating solution.

6. The core tablets were then coated with above coating solution until a weight buildup of 8 to 9% was achieved.

7. Enteric-coated tablets were further film coated using Opadry (10% w/w) suspension in water.

In Vitro Dissolution Testing Results:

Media: 0.1 N hydrochloric acid (initial 2 hours) and then a pH 7.5 TRIS [tris(hydroxymethyl)aminomethane] buffer.

Apparatus: USP type 2 [“Apparatus 2” in Test 711—Dissolution, United States Pharmacopeia 24, United States Pharmacopeial Convention, Inc., Rockville, Md. U.S.A., page 1942 (2000)].

Stirring speed: 150 rpm.

Volume: 750 mL for acid and 1000 mL for TRIS buffer.

Temperature: 37.5±0.5° C.

| | Cumulative % Drug Released | |
|---|---|---|
| | PAXIL ® CR Tablets | Example 2 Paroxetine 37.5 mg |
| Time (hours) | 37.5 mg | CR Tablets |
| 2 | 0 | 0 |
| 4 | 19 | 25 |
| 6 | 49 | 62 |
| 8 | 74 | 83 |
| 10 | 87 | 90 |

### EXAMPLE 3

### Comparative Stability Study

Compositions prepared according to Example 1 (paroxetine CR tablets 12.5 mg) and PAXIL® CR tablets 12.5 mg were stored under direct exposure to accelerated stability conditions at 40° C. and 75% relative humidity.

Percentages of paroxetine that converted to degradation products during storage are shown below:

| Sampling Time | Example 1 (12.5 mg) | Paxil ® CR 12.5 mg |
|---|---|---|
| Initial | 0.24 | 3.95 |
| 15 days | 0.27 | 4.25 |
| 1 month | 0.28 | 4.13 |

### EXAMPLE 4

### Comparative Stability Study

Compositions prepared according to Example 1 (paroxetine CR tablets 12.5 mg) and PAXIL® CR tablets 12.5 mg were packaged in sealed high-density polyethylene bottles and stored at 40° C. and 75% relative humidity.

Percentages of contained paroxetine that converted to degradation products during storage are shown below:

| Sampling Time | Example 1 (12.5 mg) | Paxil ® CR 12.5 mg |
|---|---|---|
| Initial | 0.24 | 3.95 |
| 2 months | 0.23 | 4.36 |
| 3 months | 0.51 | 6.44 |

### EXAMPLES 5 AND 6

### Compositions for Paroxetine 37.5 mg CR Tablets

| | Quantity (g) | | | |
|---|---|---|---|---|
| | Example 5 | | Example 6 | |
| Ingredient | (4000 tablets) | | (1000 tablets) | |
| Paroxetine hydrochloride hemihydrate | 175.2 | | 43.8 | |
| Ethylcellulose 100 cP | 360 | | 5 | |
| Hydroxypropyl methylcellulose 15 cP | 360 | | 75 | |
| (Methocel ™ E15) | | | | |
| Glyceryl behenate | — | | 5 | |
| Mannitol | — | | 15 | |
| Lactose | — | | 13.8 | |
| Magnesium stearate | — | | 0.8 | |
| Colloidal silicon dioxide | — | | 1.6 | |
| Tribasic calcium phosphate | 98.2 | | — | |
| Magnesium stearate | 12 | | — | |
| Core weight (a) | 250 | mg | 160 | mg |
| Eudragit L100-55 | 115.5 | | 115.5 | |
| Triethyl citrate | 11.6 | | 11.6 | |
| Talc | 16.5 | | 16.5 | |
| Isopropyl alcohol* | 1500 | | 1500 | |
| Enteric coating weight (b) | 18 | mg | 18 | mg |
| Opadry YS-1-106134 (c) | 6 | mg | 6 | mg |
| Finished tablet weight (a + b + c) | 274 | mg | 184 | mg |

*Evaporates during storage.

Manufacturing Process:

1. Paroxetine and excipients of the core were mixed together by blending.

2. The blend of step 1 was compressed into tablets.

3. Enteric-coating solution was prepared by dissolving Eudragit L 100-55 in isopropyl alcohol (8% w/w). Further, triethyl citrate and talc were added to the coating solution.

4. The core tablets of step 2 were then coated with coating solution of step 3 until a weight buildup of 8 to 9% was achieved.

In Vitro Dissolution Testing Results for Example 5 Tablets:

Media: 0.1 N hydrochloric acid (initial 2 hours) and then a pH 7.5 TRIS buffer.

Apparatus: USP type 2.

Stirring speed: 150 rpm.

Volume: 750 mL for acid and 1000 mL for TRIS buffer.

Temperature: 37.5±0.5° C.

| Time (hours) | Cumulative % Drug Released |
|---|---|
| 2 | 0 |
| 4 | 30 |
| 6 | 71 |
| 8 | 99 |
| 10 | 101 |

### EXAMPLE 7

### Composition for Paroxetine 37.5 mg Controlled Release Tablets

| | Quantity | |
|---|---|---|
| | for 1000 | |
| Ingredient | Tablets (g) | |
| Paroxetine hydrochloride hemihydrate | 43.8 | |
| Hydroxypropyl methylcellulose 100,000 cP | 8.4 | |
| (Methocel ™ K100M) | | |
| Hydroxypropyl methylcellulose 15 cP | 14 | |
| (Methocel ™ E15) | | |
| Glyceryl behenate | 4.2 | |
| Sodium lauryl sulfate | 0.2 | |
| Polysorbate 80 | 0.2 | |
| Lactose monohydrate | 45.8 | |
| Mannitol | 21.4 | |
| Colloidal silicon dioxide | 1.4 | |
| Magnesium stearate | 0.7 | |
| Isopropyl alcohol* | 450 | |
| Water* | 150 | |
| Core weight (a) | 140 | mg |
| Eudragit L100-55 | 13.5 | |
| Triethyl citrate | 2.7 | |
| Talc | 1.8 | |
| Isopropyl alcohol* | 1500 | |
| Enteric coating weight (b) | 18 | mg |
| Opadry YS-1-106134 (c) | 6 | |
| Finished tablet weight (a + b + c) | 164 | mg |

*Evaporates during processing.

Manufacturing Process:

1. Paroxetine hydrochloride, hydroxypropylmethyl cellulose, glyceryl behenate, mannitol, lactose, sodium lauryl sulfate, and polysorbate were passed through a 40 mesh sieve and blended together using a double cone blender.

2. The blend was granulated using the mixture of isopropyl alcohol and water.

3. The granules were dried in the oven at a temperature 55-65° C. until the loss on drying was 2% w/w when tested using an infrared moisture analyzer at a temperature of 105° C.

4. The dried granules were passed through an ASTM 60 mesh sieve.

5. Colloidal silicon dioxide and magnesium stearate were passed through an ASTM 80 mesh sieve, added to the granules containing active agent and mixed together.

6. The blend of step 5 was compressed into tablets weighing 135-145 mg (average weight per tablet 140 mg).

7. Enteric-coating solution was prepared by dissolving Eudragit L 100-55 in isopropyl alcohol (8% w/w). Further, triethyl citrate and talc were added to the coating solution.

8. The core tablets of step 6 were then coated with coating solution of step 7 until a weight buildup of 8 to 9% was achieved.

In Vitro Dissolution Testing Results:

Media: 0.1 N hydrochloric acid (initial 2 hours) and then a pH 7.5 TRIS buffer.

Apparatus: USP type 2.

Stirring speed: 150 rpm.

Volume: 750 mL for acid and 1000 mL for TRIS buffer.

Temperature: 37.5±0.5° C.

Cumulative % Drug Released

| Time | Paxil ® CR 37.5 mg | Example 7 Paroxetine |
|---|---|---|
| (hours) | Tablets | 37.5 mg CR Tablets |
| 2 | 0 | 0 |
| 4 | 19 | 18 |
| 6 | 49 | 64 |
| 8 | 74 | 90 |
| 10 | 87 | 97 |

In Vivo Bioavailability Testing Results:

An in vivo study was carried out to compare the paroxetine controlled release tablets (37.5 mg) with the same strength commercial PAXIL® product in twenty human subjects under a fasting state, using a single-dose 2-way crossover study design. Results are in the following table, where values are mean ±coefficient of variation (%) for the 20 subjects:

| | Example 7 | |
|---|---|---|
| Pharmacokinetic | Paroxetine 37.5 mg | PAXIL ® 37.5 mg |
| Parameter | CR Tablets | CR Tablets |
| Cₘₐₓ (ng/ml) | 17 ± 55 | 8.1 ± 83.8 |
| AUC₍₀₋ₜ₎ (ng · hour/ml) | 441.4 ± 78.4 | 188.5 ± 106.4 |
| AUC_{(0-∞)} (ng · hour/ml) | 464.3 ± 77.7 | 203.6 ± 105.4 |

### EXAMPLES 8 AND 9

### Compositions for Paroxetine 12.5 mg and 25 mg CR Tablets

Quantity for
1000 Tablets (g)

| | | |
|---|---|---|
| | Example 8 | Example 9 |
| | (12.5 mg | (25 mg |
| Ingredient | Strength) | Strength) |

| | | | | |
|---|---|---|---|---|
| Paroxetine hydrochloride hemihydrate | 14.6 | | 29.2 | |
| Hydroxypropyl methylcellulose | 8.4 | | 8.4 | |
| 100,000 cP (Methocel ™ K100M) | | | | |
| Hydroxypropyl methylcellulose 15 cP | 14.1 | | 14 | |
| (Methocel ™ E15) | | | | |
| Glyceryl behenate | 4.2 | | 4.2 | |
| Polysorbate 80 | 0.1 | | 0.1 | |
| Sodium lauryl sulfate | 0.1 | | 0.1 | |
| Lactose monohydrate | 40 | | 48.5 | |
| Mannitol | 17.4 | | 19.3 | |
| Colloidal silicon dioxide | 0.1 | | 0.1 | |
| Magnesium stearate | 0.1 | | 0.1 | |
| Isopropyl alcohol* | 15 | | 20 | |
| Water* | 75 | | 100 | |
| Core weight (a) | 100 | mg | 124 | mg |
| Eudragit L100-55 | 20 | | 20 | |
| Triethyl citrate | 2 | | 2 | |
| Talc | 1.4 | | 1.4 | |
| Isopropyl alcohol* | 293 | | 293 | |
| Enteric coating weight (b) | 14 | mg | 14 | mg |
| Opadry YS-1-106134 (c) | 4 | | 4 | |
| Finished tablet weight (a + b + c) | 118 | mg | 142 | mg |

*Evaporates during processing.

Manufacturing Process:

1. Paroxetine hydrochloride, hydroxypropyl methylcellulose, glyceryl behenate, lactose monohydrate and mannitol were passed through a #40 mesh (ASTM) sieve.

2. Sieved excipients blend was mixed in a granulator for 10 minutes and granulated using a hydroalcoholic solution of sodium lauryl sulfate and polysorbate 80.

3. The granules were dried in the oven at a temperature 55-65° C. until the loss on drying was 2% w/w when tested using an infrared moisture analyzer at a temperature of 105° C.

4. Magnesium stearate and colloidal silicon dioxide were passed through an #80 mesh sieve and blended with the dried granules using a double cone blender for 10 minutes.

5. The blend was compressed into tablets.

6. Enteric-coating solution was prepared by dissolving Eudragit L 100-55 in isopropyl alcohol (8% w/w). Further, triethyl citrate and talc were added to the coating solution.

7. The core tablets of step 5 were then coated with coating solution of step 6 until the weight buildup of 12 to 14% was achieved using pan-coating equipment.

8. Enteric-coated tablets were further film coated using a Opadry (10% w/w) suspension in water using pan-coating equipment.

In Vitro Dissolution Testing Results:

Media: 0.1 N hydrochloric acid (initial 2 hours) and then a pH 7.5 TRIS buffer.

Apparatus: USP type 2.

Stirring speed: 150 rpm.

Volume: 750 mL for acid and 1000 mL for TRIS buffer.

Temperature: 37.5±0.5° C.

| | Cumulative % Drug Released | | | |
|---|---|---|---|---|
| Time | | PAXIL ® 12.5 mg | | PAXIL ® 25 mg |
| (hours) | Example 8 | CR Tablets | Example 9 | CR Tablets |
| 2 | 0 | 0 | 1 | 0 |
| 4 | 24 | 27 | 17 | 19 |
| 6 | 67 | 58 | 63 | 46 |
| 8 | 93 | 81 | 97 | 70 |
| 10 | 95 | 87 | 101 | 83 |

### EXAMPLES 10 AND 11

### Compositions for Paroxetine 37.5 mg CR Tablets

| | mg/Tablet | |
|---|---|---|
| Ingredient | Example 10 | Example 11 |
| Paroxetine hydrochloride hemihydrate | 42.7 | 42.7 |
| Lactose monohydrate | 170 | 170 |
| Dicalcium phosphate (Di-Tab) | 27.55 | 22.55 |
| Hydroxypropyl methylcellulose 100 cP | 25 | — |
| (Methocel ™ K100 LV) | | |
| Hydroxypropyl methylcellulose 4,000 cP | 30 | 20 |
| (Methocel ™ K4M) | | |
| Hydroxypropyl methylcellulose 10,000 cP | — | 40 |
| (Methocel ™ E10M) | | |
| Colloidal silicon dioxide (Aerosil) | 2.5 | 2.5 |
| Magnesium stearate | 2.25 | 2.25 |
| Isopropyl alcohol* | 36 | 36 |
| Water* | 4 | 4 |

Enteric Coating

| | | |
|---|---|---|
| Eudragit L100-55 | 22.5 | 22.5 |
| Triethyl citrate | 3 | 3 |
| Talc | 4 | 4 |
| Titanium dioxide | 0.2 | 0.2 |
| FDC Blue No. 2 | 0.3 | 0.3 |
| Isopropyl alcohol* | 345 | 345 |
| Finished tablet weight | 330 | 330 |

*Evaporates during processing

Manufacturing Process:

1. Paroxetine HCl and lactose monohydrate were sifted through a #30 mesh sieve and mixed well, then the blend was granulated using a mixture of isopropyl alcohol and water (9:1 ratio).

2. The wet mass of step 1 was passed through a #24 mesh sieve and dried at 45° C.

3. The dried granules of step 2 were passed through a #30 mesh sieve and transferred to a double cone blender.

4. Dicalcium phosphate and both grades of hydroxypropyl methylcellulose were sifted through a #30 mesh sieve, Aerosil was sifted through a # 40 mesh sieve, and all sifted materials were added to the granules of step 3 and mixed for 10 minutes.

5. Magnesium stearate was sifted through a #40 mesh sieve, added to the blend of step 4 in the double cone blender and mixed for 3 minutes.

6. The lubricated blend of step 5 was compressed into tablets.

7. Core tablets of step 6 were coated using a coating dispersion containing Eudragit L00 55, triethyl citrate, talc, titanium dioxide & FDC Blue No. 2 in isopropyl alcohol.

### EXAMPLE 12

### Composition for Paroxetine 37.5 mg CR Tablets

| Ingredient | mg/Tablet |
|---|---|
| Paroxetine hydrochloride hemihydrate | 42.7 |
| Lactose monohydrate | 50 |
| Dicalcium phosphate (Di-Tab) | 103.55 |
| Hydroxypropyl methylcellulose 15 cP | 30 |
| Hydroxypropyl methylcellulose 4,000 cP | 20 |
| (Methocel ™ K4M) | |
| Colloidal silicon dioxide (Aerosil) | 2.5 |
| Magnesium stearate | 1.25 |
| Isopropyl alcohol* | 13.5 |
| Water* | 3.5 |

Enteric Coating

| | |
|---|---|
| Eudragit L100-55 | 22.5 |
| Triethyl citrate | 4.5 |
| Talc | 3 |
| Isopropyl alcohol* | 345 |

Film coating

| | |
|---|---|
| Opadry Blue | 5 |
| Water* | 45 |
| Finished tablet weight | 285 |

*Evaporates during processing.

Manufacturing Process:

1. Paroxetine HCl and lactose monohydrate were sifted through a #30 mesh sieve and mixed well, then the blend was granulated using a mixture of isopropyl alcohol and water (9:1 ratio).

2. The wet mass of step 1 was passed through a #24 mesh sieve and dried at 45° C.

3. The dried granules of step 2 were passed through a #30 mesh sieve and transferred to a double cone blender.

4. Dicalcium phosphate and both grades of hydroxypropyl methylcellulose were sifted through a #30 mesh sieve, Aerosil was sifted through a #40 mesh sieve, and the sifted materials were added to the granules of step 3 and mixed for 10 minutes.

5. Magnesium stearate was sifted through a #40 mesh sieve, added to the blend of step 4 in a double cone blender and mixed for 3 minutes.

6. The lubricated blend of step 5 was compressed into tablets.

7. Core tablets of step 6 were coated using a coating dispersion containing Eudragit L00 55, triethyl citrate and talc in isopropyl alcohol.

8. Enteric coated tablets of step 7 were coated with a Opadry Blue dispersion in water.

### EXAMPLE 13

### Composition for Paroxetine 37.5 mg CR Tablets

| Ingredient | mg/Tablet |
|---|---|
| Paroxetine hydrochloride hemihydrate | 42.7 |
| Lactose monohydrate | 170 |
| Dicalcium phosphate (Di-Tab) | 27.55 |
| Hydroxypropyl methylcellulose 100,000 cP | 15 |
| (Methocel ™ K100M) | |
| Hydroxypropyl methylcellulose 4,000 cP | 25 |
| (Methocel ™ K4M) | |
| Colloidal silicon dioxide (Aerosil) | 2.5 |
| Magnesium stearate | 2.25 |
| Isopropyl alcohol* | 36 |
| Water* | 4 |

Enteric Coating

| | |
|---|---|
| Eudragit L100-55 | 22.5 |
| Triethyl citrate | 3 |
| Talc | 4 |
| Titanium dioxide | 0.2 |
| FDC Blue No. 2 | 0.3 |
| Isopropyl alcohol* | 345 |
| Finished tablet weight | 315 |

*Evaporates during processing.

Manufacturing process: the composition is prepared by the process described for Examples 10 and 11.

We claim:

1. A pharmaceutical composition comprising:
a) a compressed core containing a mixture comprising:
paroxetine or a salt thereof, ethylcellulose, and a hydroxypropyl methylcellulose polymer having a nominal viscosity about 5 to about 100 cP; or
paroxetine or a salt thereof and a combination of a hydroxypropyl methylcellulose polymer having a nominal viscosity about 25,000 to about 100,000 cP and a hydroxypropyl methylcellulose polymer having a nominal viscosity about 4,000 to about 15,000 cP; or
paroxetine or a salt thereof and a combination of different hydroxypropyl methylcellulose polymers having nominal viscosities about 4,000 to about 15,000 cP; or
paroxetine or a salt thereof and a combination of a hydroxypropyl methylcellulose polymer having a nominal viscosity about 25,000 to about 100,000 cP and a hydroxypropyl methylcellulose polymer having a nominal viscosity about 5 to about 100 cP; and
b) a coating over the core comprising an acid-resistant polymer.

2. The pharmaceutical composition of claim 1, wherein paroxetine is present in the form of a salt.

3. The pharmaceutical composition of claim 1, wherein ethylcellulose has a nominal viscosity about 100 cP.

4. The pharmaceutical composition of claim 1, wherein hydroxypropyl methylcellulose that is mixed with ethylcellulose has a viscosity about 15 cP.

5. The pharmaceutical composition of claim 1, wherein a mixture comprising paroxetine or a salt thereof, ethylcellulose, and a hydroxypropyl methylcellulose polymer comprises about 10 to about 45 weight percent ethylcellulose and about 25 to about 50 weight percent hydroxypropyl methylcellulose polymer.

6. The pharmaceutical composition of claim 1, wherein a mixture comprising paroxetine or a salt thereof, ethylcellulose, and a hydroxypropyl methylcellulose polymer comprises about 10 to about 20 weight percent ethylcellulose and about 25 to about 35 weight percent hydroxypropyl methylcellulose polymer.

7. The pharmaceutical composition of claim 1, wherein a mixture comprising paroxetine or a salt thereof and a combination of hydroxypropyl methylcellulose polymers comprises about 5 to about 10 weight percent of a hydroxypropyl methylcellulose polymer having a nominal viscosity about 25,000 to about 100,000 cP and about 8 to about 15 weight percent of a hydroxypropyl methylcellulose polymer having a nominal viscosity about 5 to about 100 cP.

8. The pharmaceutical composition of claim 1, wherein a mixture comprising paroxetine or a salt thereof and a combination of hydroxypropyl methylcellulose polymers comprises about 5 to about 10 weight percent of a hydroxypropyl methylcellulose polymer having a nominal viscosity about 100,000 cP and about 8 to about 15 weight percent of a hydroxypropyl methylcellulose polymer having a nominal viscosity about 15 cP.

9. The pharmaceutical composition of claim 1, wherein a mixture comprising paroxetine or a salt thereof and a combination of hydroxypropyl methylcellulose polymers comprises about 3 to about 8 weight percent of a hydroxypropyl methylcellulose polymer having a nominal viscosity about 25,000 to about 100,000 cP and about 5 to about 10 weight percent of a hydroxypropyl methylcellulose polymer having a nominal viscosity about 4,000 to about 15,000 cP.

10. The pharmaceutical composition of claim 1, wherein a mixture comprising paroxetine or a salt thereof and a combination of hydroxypropyl methylcellulose polymers comprises about 3 to about 8 weight percent of a hydroxypropyl methylcellulose polymer having a nominal viscosity about 100,000 cP and about 5 to about 10 weight percent of a hydroxypropyl methylcellulose polymer having a nominal viscosity about 4,000 cP.

11. The pharmaceutical composition of claim 1, wherein a mixture comprising paroxetine or a salt thereof and a combination of different hydroxypropyl methylcellulose polymers having nominal viscosities about 4,000 to about 15,000 cP comprises about 5 to about 15 weight percent of each polymer.

12. The pharmaceutical composition of claim 1, wherein a mixture comprising paroxetine or a salt thereof and a combination of hydroxypropyl methylcellulose polymers comprises about 5 to about 10 weight percent of a hydroxypropyl methylcellulose polymer having a nominal viscosity about 4,000 cP and about 8 to about 15 weight percent of a hydroxypropyl methylcellulose polymer having a nominal viscosity about 10,000 cP.

13. A pharmaceutical composition comprising:
a compressed core containing paroxetine or a salt thereof, a hydroxypropyl methylcellulose polymer, and an ethylcellulose polymer; and
a coating over the core comprising an acid-resistant polymer.

14. The pharmaceutical composition of claim 9, wherein a hydroxypropyl methylcellulose polymer comprises about 25 to about 50 weight percent of the core and an ethylcellulose polymer comprises about 10 to about 45 weight percent of the core.

15. The pharmaceutical composition of claim 9, wherein a hydroxypropyl methylcellulose polymer and an ethylcellulose polymer each comprise about 35 to about 40 weight percent of the core.

16. The pharmaceutical composition of claim 9, wherein a hydroxypropyl methylcellulose polymer comprises about 25 to about 35 weight percent of the core and an ethylcellulose polymer comprises about 10 to about 20 weight percent of the core.

17. The pharmaceutical composition of claim 9, wherein a hydroxypropyl methylcellulose polymer has a nominal viscosity about 5 to about 100 cP.

18. The pharmaceutical composition of claim 9, wherein ethylcellulose has a nominal viscosity about 100 cP.

19. A pharmaceutical composition comprising:
a compressed core containing paroxetine or a salt thereof and a combination of a hydroxypropyl methylcellulose polymer having a nominal viscosity about 100,000 cP and a hydroxypropyl methylcellulose polymer having a nominal viscosity about 15 cP; and
a coating over the core comprising an acid-resistant polymer.

20. The pharmaceutical composition of claim 15, wherein a hydroxypropyl methylcellulose polymer having a viscosity about 100,000 cP comprises about 6 to about 9 weight percent of the core, and a hydroxypropyl methylcellulose polymer having a viscosity about 15 cP comprises about 8 to about 15 weight percent of the core.

21. The pharmaceutical composition of claim 15, wherein at least about 60 percent of total contained paroxetine is released within about 4 hours, and at least about 90 percent of total contained paroxetine is released within about 6 hours, during immersion in an aqueous fluid having a pH about 7.5 using a United States Pharmacopeia type 2 dissolution test apparatus.

22. The pharmaceutical composition of claim 15, which upon administration of a single dose containing 37.5 mg of paroxetine to human subjects provides a mean Cₘₐₓ at least about 15 ng/ml and a mean AUC at least about 400 ng-hour/ml.

23. A pharmaceutical composition comprising:
a compressed core containing paroxetine or a salt thereof and a combination of different hydroxypropyl methylcellulose polymers having nominal viscosities about 4,000 to about 15,000 cP; and
a coating over the core comprising an acid-resistant polymer.

24. A pharmaceutical composition comprising:
a compressed core containing paroxetine or a salt thereof and a combination of a hydroxypropyl methylcellulose polymer having a nominal viscosity about 4,000 to about 15,000 cP and a hydroxypropyl methylcellulose polymer having a nominal viscosity about 25,000 to about 100,000 cP; and
a coating over the core comprising an acid-resistant polymer.

25. A pharmaceutical composition comprising:
a compressed core containing paroxetine or a salt thereof and a combination of hydroxypropyl methylcellulose polymers comprising about 5 to about 10 weight percent of a hydroxypropyl methylcellulose polymer having a nominal viscosity about 4,000 to about 15,000 cP and about 4 to about 15 weight percent of a hydroxypropyl methylcellulose polymer having a nominal viscosity about 5 to about 100 cP; and
a coating over the core comprising an acid-resistant polymer.

26. The pharmaceutical composition of claim 25, wherein a compressed core containing paroxetine or a salt thereof comprises about 5 to about 10 weight percent of a hydroxypropyl methylcellulose polymer having a nominal viscosity about 4,000 cP and about 4 to about 15 weight percent of a hydroxypropyl methylcellulose polymer having a nominal viscosity about 100 cP.
